(19) United States
(12) Patent Application Publication
(10) Pub. No.: US 2005/0222605 A1
(43) Pub. Date: Oct. 6, 2005

(54) Occluding coil delivery device and method

(75) Inventors: E. Skott Greenhalgh, Wyndmoor, PA (US); Michael P. Igoe, Perkasie, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Secant Medical, LLC, Perkasie, PA (US)

(21) Appl. No.: 11/079,753

(22) Filed: Mar. 14, 2005

Related U.S. Application Data
(60) Provisional application No. 60/553,518, filed on Mar. 16, 2004.

(51) Int. Cl. A61M 29/00

(52) U.S. Cl. 606/200

(57) ABSTRACT

A device for use within the lumen of a catheter to move an item positioned within the lumen relatively to the catheter is disclosed. The device is formed from an elongated member positionable within the lumen and reciprocably movable relatively to the catheter. A distal end of the member is engaged with the item, and a proximate end extends outwardly from the catheter. A gripping element is attached to the elongated member at the distal end. The gripping element has a free end that extends outwardly from the elongated member. The free end of the gripping element is engageable and disengageable with the item, the item being moved when the gripping element is engaged and the elongated member is moved relatively to the catheter.

### FIELD OF THE INVENTION

The invention concerns a device and a method for delivering occluding coils in the treatment of vascular aneurysms.

### BACKGROUND OF THE INVENTION

Vascular aneurysms occur in arteries in the body and comprise an outwardly extending sack-like formation of the artery wall. The aneurysm has a neck forming the juncture with the artery and is capped by a dome. During formation of the aneurysm, the arterial internal elastic lamina disappears at the base of the neck, the sack wall thins and weakens and connective tissue replaces smooth-muscle cells. The aneurysm tends to rupture at the dome and bleeding ensues.

Rupture of a cerebrovascular aneurysm is especially serious due to the associated high mortality rate (10% within the first day of rupture, 25% within three months) and the major neurological deficits experienced by those who survive the initial hemorrhage. Naturally, therapeutic treatment of cerebrovascular aneurysms emphasizes preventing the initial rupture.

Intravascular Occlusion Treatment Technique

Intravascular occlusion techniques for treating aneurysms are discussed in U.S. Pat. No. 5,122,136, hereby incorporated by reference, and U.S. Pat. No. 6,010,498, also hereby incorporated by reference.

The techniques described in these patents can be summarized with reference to FIGS. 1 and 2, which show a saccular aneurysm 20 formed in an artery 22 at a bifurcation point 24. The treatment techniques involve positioning a catheter 26 at the artery bifurcation point 24, the catheter tip 28 extending partially into the neck 30 of the aneurysm 20. Once the catheter is in position, a length of wire coil 32, preferably coiled platinum or platinum alloy wire covered with fabric, is pushed through the catheter's lumen 34 through the aneurysm neck 30 and into the aneurysm 20. The coil 32 is flexible and is biased to expand to a larger size upon release from the catheter. Being flexible, the expanded coil loops and tangles randomly as it is packed into the aneurysm. Blood which would normally circulate under pressure into the aneurysm, causing it to enlarge, weaken and rupture, begins to form clots 36 on the expanded coil tangle and eventually the clots merge and enlarge to form an occlusion 38 (see FIG. 2) which seals off the aneurysm from the blood flow, preventing further enlargement and rupture.

Once the appropriate length of coil 32 is positioned in the aneurysm and the occlusion has been formed, the coil 32 is released at or near the neck 30 of the aneurysm and the catheter is withdrawn (FIG. 2). Wire release is effected by any one of several means, for example, mechanical means or electrolytic means.

While this occlusion technique holds great promise of effective treatment for preventing aneurysm rupture, especially cerebrovascular aneurysms, it is often difficult to push the coil through the lumen of the catheter over any significant distance. As noted above, the coil is biased to expand upon release from the catheter and therefore, when packed within the catheter, it pushes outwardly against the inner wall of the lumen, increasing the friction between the coil and the catheter and hindering the motion of the coil. Such coils may be between 10 cm and 20 cm long and require relatively great force be exerted to move their length through the lumen, the pushing force being in proportion to the length of coil being moved. In many cases, the coil is damaged during the delivery to the aneurysm due to the significant pushing force that it experiences. It would be advantageous to employ a device and a method that reduce the force required to deliver a coil to an aneurysm and thereby mitigate damage to the coil.

### SUMMARY OF THE INVENTION

The invention concerns a device for use within a lumen of a catheter to move an occlusion coil or other item positioned within the lumen through the catheter. The device comprises an elongated member positionable within the lumen and reciprocably movable relatively to the catheter. A gripping element is attached to the elongated member. The gripping element is positionable in spaced relation to the catheter tip and is engageable with and disengageable from the coil. Upon engagement of the gripping element with the coil, a portion of the coil positioned between the gripping element and the catheter tip is pushed out of the lumen upon motion of the elongated member toward the catheter tip. Upon disengagement of the gripping element from the coil, the elongated member may be moved relatively to the catheter without moving the coil. Reciprocal motion of the elongated member, with the gripping element alternatingly engaging and disengaging the coil moves the coil through the catheter.

There are a number of embodiments that may comprise the gripping element. For example, the gripping element may comprise a plurality of flexible appendages that extend outwardly from the elongated member. Each of the flexible appendages is angularly oriented relatively to the elongated member and have a free end facing the catheter tip and an opposite end attached to the elongated member.

In another embodiment, the gripping element comprises an elastic body mounted on the elongated member. The elastic body may comprise a balloon or an elastic tube. The tube has first and second ends, the first end of the tube facing the catheter end, the second end of the tube being attached to the elongated member.

The gripping element may also comprise a braided tube having first and second ends, the first end of the tube facing the catheter tip, the second end of the tube being attached to the elongated member.

Alternately, the gripping element may comprise a coil spring having first and second ends, the first end of the spring facing the catheter tip, the second end of the spring being attached to the elongated member.

The gripping element may also be in the form of a cone having first and second ends, the first end having a relatively larger diameter than the second end, the first end of the cone facing the catheter tip, the second end of the cone being attached to the elongated member.

In another embodiment, the gripping element may comprise one or more hooks mounted on the elongated member and oriented so as to engage the coil when the elongated member is moved in one direction, for example, toward the catheter tip, the hooks disengaging from the coil upon motion of the elongated member in the opposite direction.

Furthermore, the gripping element may comprise projections or appendages that can be mechanically or hydraulically extended from or retracted toward or into the elongated member.

The invention also includes a method of delivering a coil from a catheter into an aneurysm. The method comprises the steps of:

(A) providing a catheter having a coil positioned therein, an elongated member positioned therein, and a gripping element mounted on the elongated member;

(B) positioning the catheter tip within the aneurysm;

(C) engaging the gripping element with the coil;

(D) moving the elongated member toward the catheter tip, a portion of the coil positioned between the gripping element and the catheter tip being pushed out from the catheter tip and into the aneurysm;

(E) disengaging the gripping element from the coil;

(F) moving the elongated member and thereby the gripping element away from the catheter tip; and

(G) repeating steps C-F as required to move the coil into the aneurysm.

### BRIEF DESCRIPTION OF THE DRAWINGS

FIGS. 1 and 2 are partial longitudinal sectional views illustrating the treatment of a vascular aneurysm;

FIGS. 3-5 are partial longitudinal sectional views illustrating a device and a method for delivering an occluding coil by means of a catheter for the treatment of vascular aneurysms; and

FIGS. 6-15 illustrate various embodiments of a device for pushing an occluding coil from a catheter in the treatment of vascular aneurysms.

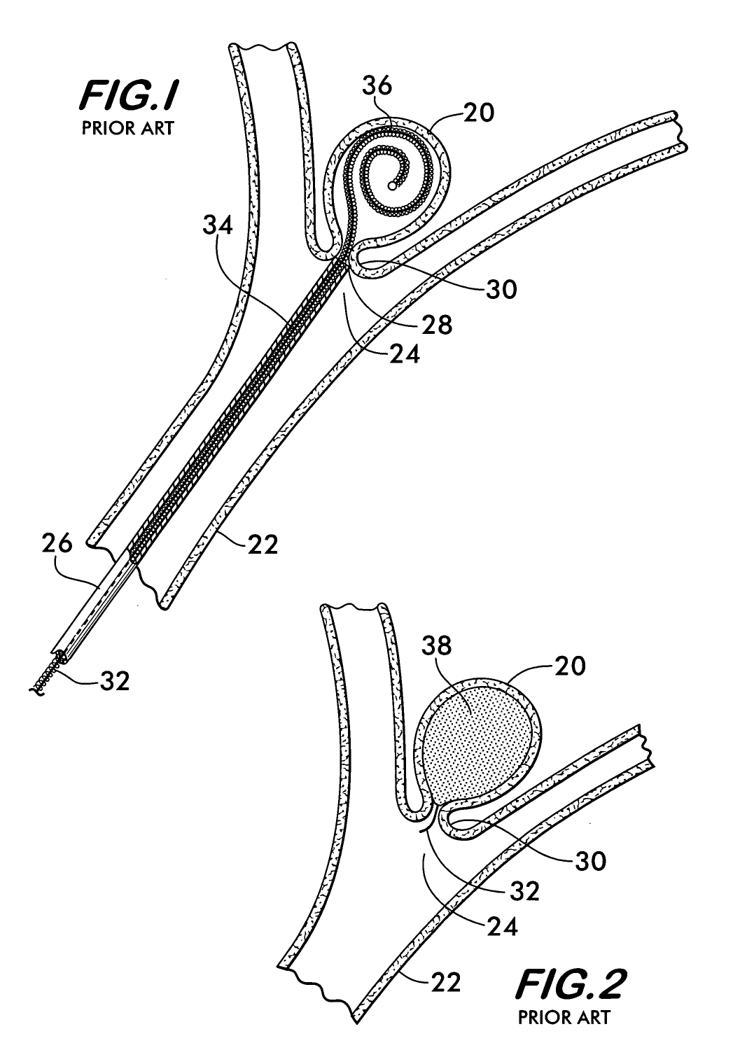

### DETAILED DESCRIPTION OF THE PRESENTLY PREFERRED EMBODIMENTS

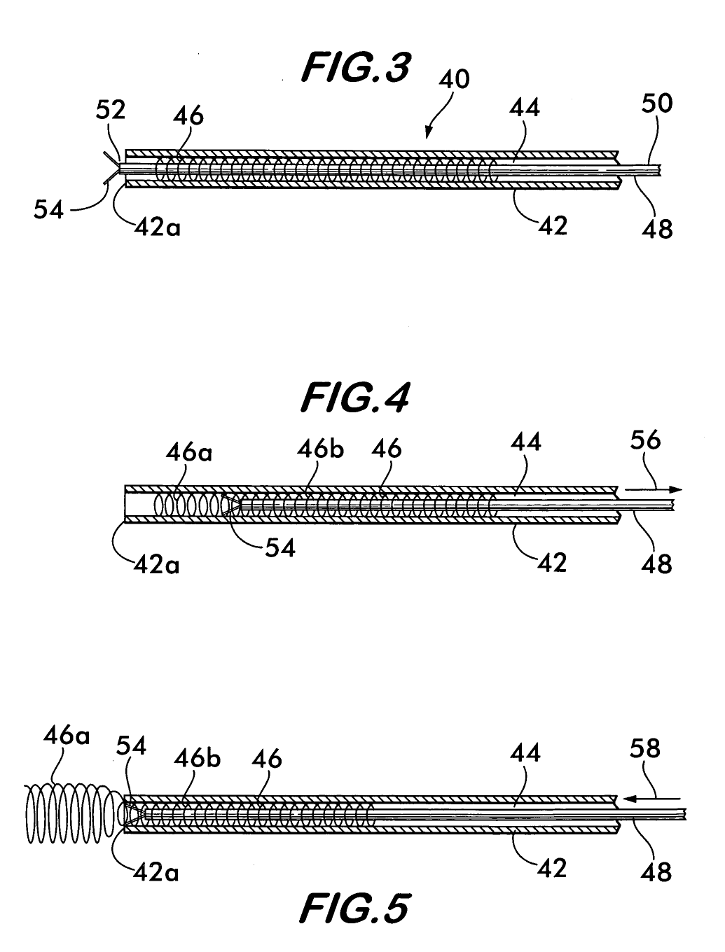

The method and device 40 for delivering an occluding coil in the treatment of vascular aneurysms according to the invention is described with reference to FIGS. 3-5.

As shown in FIG. 3, device 40 comprises a catheter 42 positionable within a vascular vessel. Catheter 42 has a lumen 44 in which an occluding coil 46 is packed. As described above, coil 46 preferably comprises a platinum or platinum alloy wire covered with fabric, the wire being biased to assume an expanded helical shape in the absence of restraining forces. Being flexible and resilient, the coil 46 may be readily deformed into a smaller volume so as to fit within lumen 44 so that it may traverse a vascular vessel, expanding by itself upon release from lumen 44 into an aneurysm to form a structure upon which blood may clot to form an occlusion. Preferably, the coil 46 is pre-positioned near the tip 42a of catheter 42 so that it need not traverse the entire length of the catheter to be released into the aneurysm.

An elongated member 48 is also positioned within lumen 44 of catheter 42. Elongated member 48 is flexible and is preferably positioned coaxially within the lumen 44, surrounded by coil 46. Elongated member 48 has a proximal end 50 that extends out from the catheter 42. Proximal end 50 allows the elongated member 48 to be manually moved relatively to the coil and the catheter. Opposite proximal end 50, elongated member 48 has a distal end 52 to which is attached a gripping element 54. The particular design of the gripping element is described in detail below. Gripping element 54 is adapted to engage with or disengage from coil 46.

The gripping element 54 cooperates with the elongated member 48 to push the coil 46 from the lumen 44. As shown in FIG. 4, the gripping element 54 is disengaged from the coil 46. In this disengaged state, the elongated member 48 may be drawn in a direction indicated by arrow 56 away from catheter tip 42a to position the gripping element 54 such that there is a predetermined length of coil 46a between it and the catheter tip 42a. During motion of the gripping element 54 in the direction away from the tip 42a, the gripping element does not move the coil relatively to the catheter 42. As shown in FIG. 5, the elongated member 48 is then moved toward the catheter tip 42a in a direction indicated by arrow 58. As elongated element 48 is moved toward the catheter tip, the gripping element 54 engages the coil 46, forcing at least a portion of the predetermined coil length 46a outwardly from the catheter tip 42a, the coil length 46a expanding as it exits the lumen 44 due to its resilient, elastic biasing. As it pushes the coil length 46a along catheter 42, the gripping element 54 also drags another length 46b of coil 46 closer to the catheter tip 42a. This process is then repeated until the coil 46 is pushed out from the lumen 44. There is an advantage realized using the apparatus and method steps as described above in that the force required to move the relatively short coil lengths 46a and 46b along the catheter 42 with each reciprocal movement of the elongated member 48 is significantly lower than the force that would be required to push the entire coil 46 out from the lumen 44 with one motion of the elongated member pushing from the proximal end 60 of coil 46. Lowering the pushing force provides greater control over the motion of coil 46 and also decreases the chance that the coil will be damaged as it is moved through the catheter 42.

There are various embodiments of the gripping element 54 that will effectively engage the coil 46 to move it relatively to catheter 42 as well as readily disengage from the coil to allow the flexible element 54 and the elongated member 48 to move relatively to the coil 46 and the catheter 42 without moving the coil relatively to the catheter.

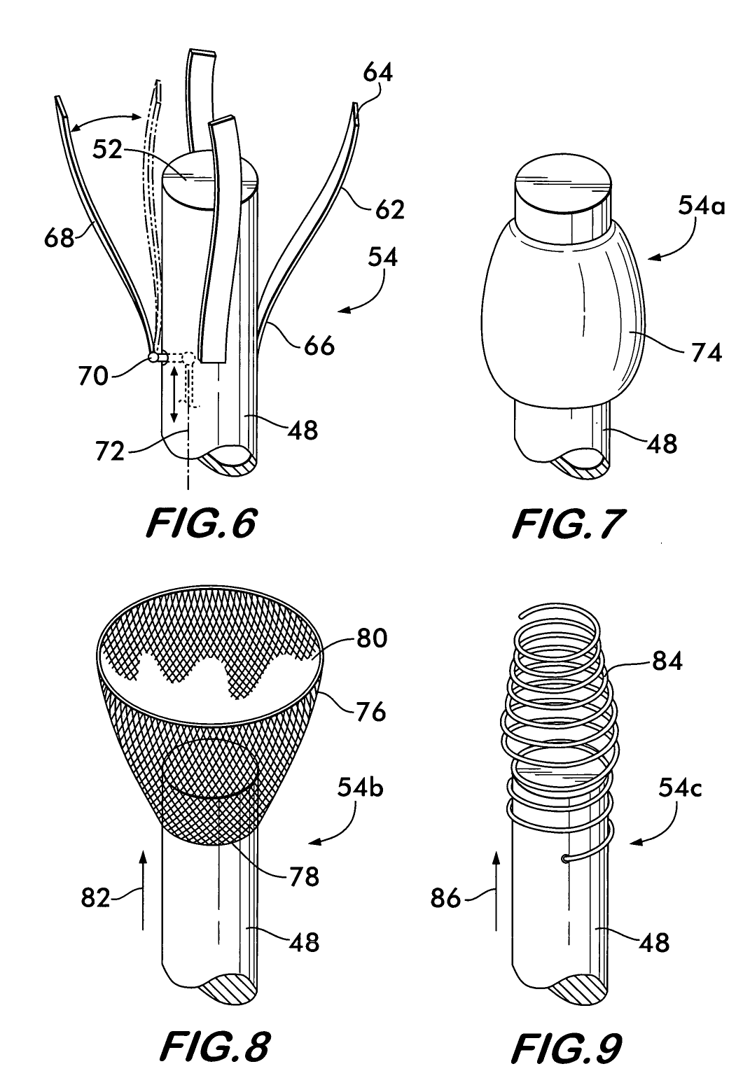

As shown in FIG. 6, gripping element 54 may comprise one or more flexible appendages 62 preferably attached near or at the distal end 52 of the elongated member 48. Each flexible appendage 62 has a free end 64 that faces catheter tip 42a, and an opposite end 66 attached to the elongated member 48. Appendages 62 are angularly oriented relatively to the elongated member and resiliently biased to maintain contact with the coil 46 surrounding the elongated member within lumen 44. When the elongated member 48 is moved toward the catheter tip 42a, the free ends 64 engage the coil 46, and the appendages 62, due to their angular orientation, expand outwardly to engage and push the coil length 46a along lumen 44 and outwardly from catheter 42 while pulling the other coil length 46b through the lumen 44 toward the tip 42a. Again, due to their angular orientation, the appendages 62 contract inwardly when the elongated member 48 is moved away from catheter tip 42a. The free ends 64 of the appendages 62 disengage from the coil 46 and do not move it relatively to the catheter 42 when the elongated member is moved away from the catheter tip.

In the example shown in FIG. 6, the appendages 62 are resiliently biased into contact with the coil 46, the appendages comprising a polymer resin or a metal such as stainless steel, nitinol, elgiloy, titanium or other bio-compatible material that is resilient and flexible. However, it is also possible to pivotably attach an appendage 68 to the elongated member 48 via a hinge 70 and to deploy it outwardly or inwardly (as shown in dotted line) by a push-rod mechanism 72 or other actuation means when it is desired to engage the appendage with or disengage the appendage from the coil.

FIG. 7 shows another embodiment 54a of the gripping element. In this example, element 54a comprises a balloon 74 attached to the elongated member 48 that is inflatable and deflatable hydraulically or pneumatically. When it is desired to move coil 46 relatively to catheter 42, the balloon 74 is inflated and engages the coil. The elongated member 48 is moved relatively to the catheter and the coil, engaged by the balloon 74 is moved along with it. To move the elongated member 48 without moving the coil 46, the balloon 74 is first deflated to disengage it from the coil, thus allowing the balloon and the elongated member to move independently of the coil. The balloon embodiment has the advantage of allowing the coil to be moved in either direction along the lumen 44.

Another example of a gripping element 54b is shown in FIG. 8, the gripping element comprising a braided tube 76. Tube 76 is attached at one end 78 to the elongated member 48, the opposite end 80 being open. The tube is resiliently biased to remain open and in contact with the coil in the lumen 44. Due to the trellis effect associated with braided structures, the braided tube 76 will expand outwardly in diameter when it is under compression, i.e., when it is moved within the lumen 44 in the direction indicated by arrow 82. When it is moved and compressed by contact with the coil, the braided tube 76 expands outwardly, engages the coil and moves it along the lumen 44. When the elongated member 48 is moved in the opposite direction to arrow 80, contact between the braided tube 76 and the coil 46 applies a tensile force to the tube 76. Due again to the trellis effect, the tube 76 elongates and shrinks in diameter, disengaging itself from the coil and allowing the elongated member 48 and the tube 76 to move relative to both the coil and the catheter.

Similar behavior is evidenced by the embodiment 54c shown in FIG. 9, wherein a coil spring 84 is attached to the elongated member 48. The spring is sized and biased so that it is in contact with coil 46. For motion of spring 84 in the direction of arrow 86, frictional forces between the spring and the coil will tend to compress the spring, the spring expanding outwardly as a result, engaging the coil and dragging it along the lumen 44. Motion in the opposite direction places the spring in tension, extending the spring and reducing its diameter. When extended, the spring 84 disengages from the coil 46.

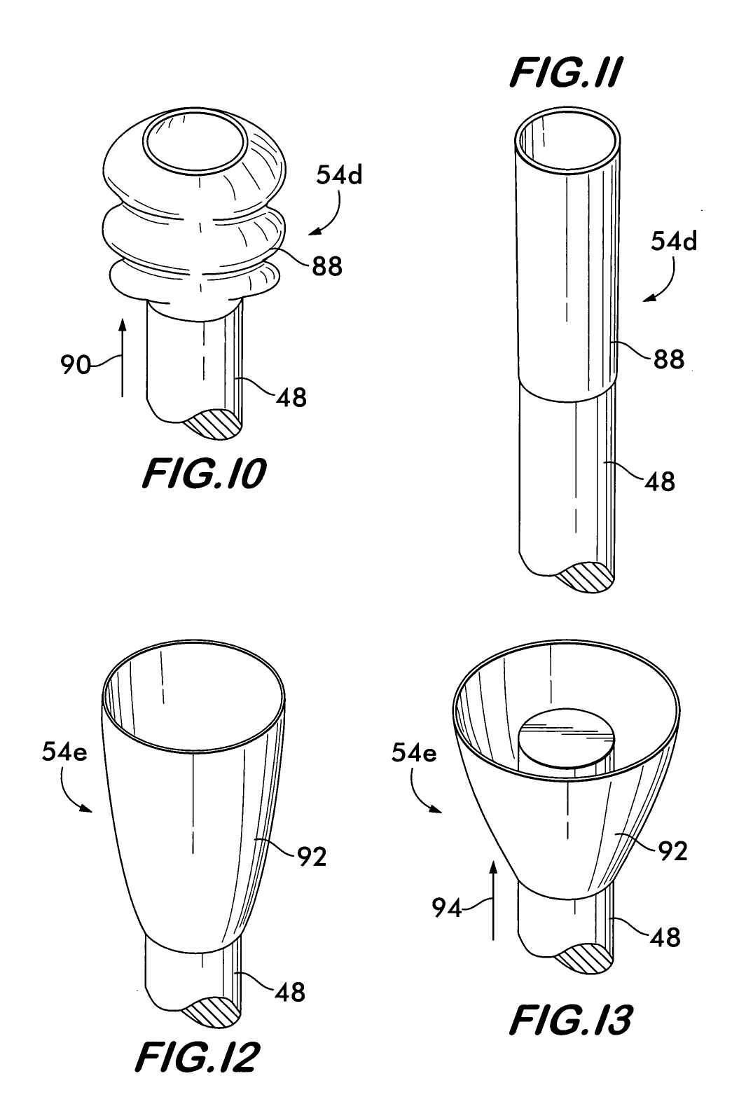

FIGS. 10 and 11 show another embodiment of gripping element 54d in the form of a flexible tube 88. Tube 88 is mounted on the elongated member 48 and sized to contact the surrounding coil 46. Moving the tube 88 in the direction of arrow 90 places the tube in compression due to friction between the tube and the coil. The tube buckles under compression and expands to engage the coil and move it along the lumen 44 as illustrated in FIG. 10. Motion in the opposite direction places the tube in tension as shown in FIG. 11. The tube lengthens and does not engage the coil with sufficient force so as to move it relatively to the catheter 42.

In another embodiment, shown in FIGS. 12 and 13, gripping element 54e may be a flexible cone 92 preferably attached at (FIG. 12) or proximate to (FIG. 13) the end of elongated member 48. The cone 92 is sized to contact the coil 46, and motion of the cone in the direction of arrow 94 compresses the cone, causing it to expand outwardly to engage and move the coil 46. If the motion is in the opposite direction, the cone merely slides over the coil and does not move it relatively to the catheter.

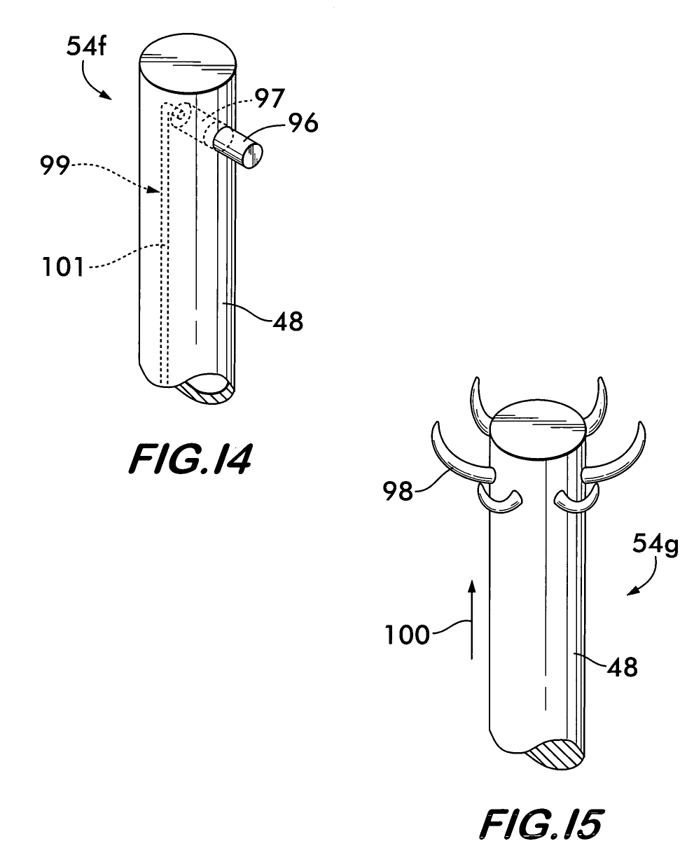

FIG. 14 shows an embodiment 54f comprising an extendible and retractable projection 96 mounted on the elongated member 48. Projection 96 may be withdrawn into a cavity 97 within the member 48, as shown in dotted line, or may extend outwardly, as shown in solid line. Extension and retraction may be accomplished by hydraulic, pneumatic or mechanical means 99 comprising, for example, a channel 101 extending lengthwise through elongated member 48. Channel 101 provides a conduit through which hydraulic or pneumatic fluid may be forced to extend and retract projection 96 from cavity 97. When it is desired to move the coil 46, the projection 96 is extended to engage it and the elongated member 48 is moved relatively to the catheter. When it is desired to move the elongated member 48 without moving the coil, the projection 96 is retracted to disengage it from the coil.

FIG. 15 illustrates another embodiment 54g having one or more hooks 98 attached to elongated member 48. Hooks 98 are oriented so that they engage the coil 46 only when the elongated member 48 is moved in the direction indicated by arrow 100. When the member 48 is moved in the opposite direction, the hooks merely slide over the coil and do not engage it, allowing elongated member 48 to move relatively to both the catheter 42 and the coil 46.

1. A device for use within a lumen of a catheter to move an item positioned within said lumen relatively to said catheter, said device comprising:
an elongated member positionable within said lumen and reciprocably movable relatively to said catheter; and
a gripping element attached to said elongated member, said gripping element being alternately engageable with and disengageable from said item, said item being movable relatively to said catheter when said gripping element is engaged with said item and said elongated member is moved, said gripping element and said elongated member being movable relatively to said catheter and said item when said gripping element is disengaged from said coil.

2. A device according to claim 1, wherein said gripping element comprises a flexible appendage extending outwardly from said elongated member, said appendage being angularly oriented relatively to said elongated member and having a free end and an opposite end attached to said elongated member.

3. A device according to claim 1, wherein said gripping element comprises an elastic body mounted on said elongated member.

4. A device according to claim 3, wherein said elastic body comprises a balloon.

5. A device according to claim 1, wherein said gripping element comprises a braided tube having first and second ends, said first end of said tube facing said catheter end, said second end of said tube being attached to said elongated member.

6. A device according to claim 1, wherein said gripping element comprises a coil spring having first and second ends, said first end of said spring facing said catheter end, said second end of said spring being attached to said elongated member.

7. A device according to claim 1, wherein said gripping element comprises an elastic tube having first and second ends, said first end of said tube facing said catheter end, said second end of said tube being attached to said elongated member.

8. A device according to claim 1, wherein said gripping element comprises a cone having first and second ends, said first end having a relatively larger diameter than said second end, said first end of said cone facing said catheter end, said second end of said cone being attached to said elongated member.

9. A device according to claim 1, wherein said gripping element comprises a hook mounted on said elongated member.

10. A device for use within a lumen of a catheter to move an item positioned within said lumen relatively to said catheter, said device comprising:
an elongated member positionable within said lumen and reciprocably movable relatively to said catheter, said elongated member having a distal end engageable with said item and a proximate end extending from said catheter;
an appendage having a first end attached to said elongated member at said distal end, and a free end oppositely disposed, said appendage being movable to position said free end outwardly from said elongated member so as to engage and grip said item for movement thereof through said lumen upon movement of said elongated member, said appendage being further movable to position said free end toward said elongated member so as to disengage said appendage from said item thereby allowing movement of said elongated member without movement of said item.

11. A device according to claim 10 having a plurality of said appendages attached to said elongated member.

12. A device according to claim 10, wherein said first end of said appendage is pivotably attached to said elongated member, said free end of said appendage facing said distal end of said elongated member, said appendage being pivotably movable to extend angularly outwardly from said elongated member.

13. A device according to claim 12, further comprising a push rod attached to said appendage, said push rod extending along and being movable relatively to said elongated member for pivoting said appendage toward and away from said elongated member.

14. A device according to claim 10, wherein said appendage comprises a projection positioned within a cavity in said distal end of said elongated member, said projection being extendable from and retractable into said cavity for engaging and disengaging with said item within said lumen.

15. A device according to claim 14, wherein said projection is extendable perpendicularly to said elongated member.

16. A device according to claim 14, further comprising a plurality of said projections.

17. A device according to claim 14, further comprising means for extending and retracting said projections mounted on said elongated member.

18. A method of delivering a coil from a catheter into an aneurysm, said method comprising the steps of:
providing a catheter having a coil positioned therein, an elongated member positioned therein, and a gripping element mounted on the elongated member;
positioning the catheter tip within the aneurysm;
engaging the gripping element with the coil;
moving the elongated member toward the catheter tip, a portion of the coil positioned between the gripping element and the catheter tip being pushed out from the catheter tip and into the aneurysm;
disengaging the gripping element from the coil;
moving the elongated member and thereby the gripping element away from the catheter tip; and
repeating the engaging, moving and disengaging steps as required to move the coil into the aneurysm.
